(19) United States
(12) Patent Application Publication
(10) Pub. No.: US 2026/0243632 A1
(43) Pub. Date: Aug. 20, 2026

(54) TISSUE SAMPLE SUPPORTS

(71) Applicant: University of Vermont and State Agricultural College, Burlington, VT (US)

(72) Inventor: Matthew A. Caporizzo, Colchester, VT (US)

(21) Appl. No.: 19/544,959

(22) Filed: Feb. 19, 2026

Related U.S. Application Data
(60) Provisional application No. 63/760,614, filed on Feb. 19, 2025.

(51) Int. Cl. G01N 1/28

(52) U.S. Cl. CPC G01N 1/286

(57) ABSTRACT

A tissue support for securing a tissue sample for analysis is described. In various embodiments, the tissue support may include a first pad and a second pad spaced apart by a predetermined distance so that, when the tissue sample is mounted, a portion of the tissue sample extends between the pads. Each pad may have an analyzer interface configured to engage a corresponding fixation point of an analyzer. One or more severable spacing members connect the pads and maintain the predetermined distance during mounting and handling, where the severable spacing member(s) are positioned laterally offset from a tissue-receiving region between the pads. After the tissue support is coupled to an analyzer, the severable spacing member(s) may be severed to allow relative movement of the pads for mechanical loading of the tissue sample and measurement of tissue response.

### CROSS-REFERENCE TO RELATED APPLICATIONS

This application claims priority to U.S. Provisional Patent Application No. 63/760,614, now pending, filed Feb. 19, 2025, the entire disclosure of which is incorporated herein by this reference.

### STATEMENT REGARDING FEDERALLY SPONSORED RESEARCH

This invention was made with government support under contract no. R01HL171614 awarded by the NIH/NHLBI. The government has certain rights in the invention.

### FIELD OF THE DISCLOSURE

The present disclosure relates generally to medical devices used in histology and tissue handling, and more particularly to tissue supports and associated methods that secure tissue samples during preparation and analysis. In various embodiments, the tissue supports facilitate mounting a tissue sample to spaced pads and coupling the mounted sample to an analyzer through analyzer interfaces so that the analyzer can apply mechanical forces to the tissue sample and/or measure tissue response.

### BACKGROUND OF THE DISCLOSURE

Histological examination of tissue samples is critical for medical diagnosis, research, understanding disease progression, etc. In particular, the analysis of myocardial tissue provides essential insights into cardiac conditions, including cardiomyopathies, infarctions, and hypertrophy. The preparation and handling of myocardial tissue samples present unique challenges due to the tissue's distinctive properties and its tendency to undergo rapid changes post-extraction.

Conventional tissue handling tools often fail to address the specific requirements of myocardial tissue while simultaneously providing a convenient form factor for use in specialized analysis equipment. These conventional tools can lead it unintended damage to the tissue structure, potentially compromising the accuracy of subsequent histological analysis. The processing of myocardial tissue samples typically involves multiple steps, and traditional handling tools often require sample transfer between different devices during these steps, increasing the risk of tissue damage or loss. Additionally, a significant challenge in myocardial tissue handling arises from the tissue's natural tendency to contract and deform after extraction. Without proper stabilization, these physical changes can alter the tissue's microscopic structure, potentially leading to artifacts in histological examination.

Recent advances in histological techniques, particularly in the field of automated tissue processing and cardiac function testing using slice systems, have created a need for more sophisticated tissue handling solutions. These modern analytical methods require samples to maintain their structural integrity throughout the preparation process, highlighting the limitations of conventional tissue handling approaches.

There exists a need for a specialized histological support designed specifically for myocardial tissue that can address these various challenges. Such a device would ideally combine features that preserve tissue structure and orientation throughout the preparation process with a convenient structure for ease of use within analysis equipment.

### BRIEF SUMMARY OF THE DISCLOSURE

The present disclosure relates to tissue supports and associated systems and methods for securing a tissue sample during preparation and for coupling the tissue sample to analysis equipment so that the tissue sample may be mechanically loaded and evaluated. In various embodiments, a tissue support includes a first pad and a second pad spaced apart by a predetermined distance so that, when the tissue sample is mounted, a portion of the tissue sample extends between the pads.

In some embodiments, each pad has an analyzer interface configured to engage a corresponding fixation point of an analyzer, such as an aperture sized to fit over a post, although other interface geometries may be used. One or more severable spacing members connect the pads and maintain the predetermined distance during mounting and handling, and the severable spacing member or members are positioned laterally offset from a region between the pads that receives the portion of the tissue sample, which facilitates severing while keeping a cutting tool outside the tissue-receiving region. In some embodiments, the severable spacing member or members are tuned by selecting material, shape, and size so that the predetermined distance is maintained despite tissue forces prior to severing, and spring-like geometries such as flexure, serpentine, or accordion shapes may be used.

In some embodiments, the tissue sample is secured to the pads using a biocompatible adhesive such as cyanoacrylate, and the tissue support material may be selected to bond with the adhesive, including embodiments that use polyacrylate to bond with cyanoacrylate. In some embodiments, at least one pad has a gripping surface with microfeatures to engage tissue. In some embodiments, the tissue support is electrically conductive, and conductive adhesive may be used so that the tissue support supports electrical stimulation and/or electrical recording through the tissue sample. In some embodiments, the tissue support is formed from a single material, and in other embodiments the pads are formed from a first material and the severable spacing member or members are formed from a second material different from the first material.

In another aspect, a tissue analysis system includes a tissue support as described above and an analyzer having fixation points that engage the analyzer interfaces, where the analyzer may include a force transducer and a length controller to apply force to the tissue sample and measure tissue response after severing the severable spacing member or members. In another aspect, a method includes attaching ends of a tissue sample to the pads, coupling the pads to the analyzer, severing the severable spacing member or members, and operating the analyzer to apply mechanical force and measure response, optionally including preparing the tissue sample with chemical agents while mounted to the tissue support.

### DESCRIPTION OF THE DRAWINGS

For a fuller understanding of the nature and objects of the disclosure, reference should be made to the following detailed description taken in conjunction with the accompanying drawings.

FIG. 1A: A perspective view of an embodiment of a tissue support according to an embodiment of the present disclosure.

FIG. 1B: A side view of the tissue support of FIG. 1A.

FIG. 1C: A top view of the tissue support of FIGS. 1A and 1B.

FIG. 2: A photograph of a tissue section and showing a tissue sample portion identified as ‘B’.

FIG. 3: A photograph of the tissue sample of FIG. 2 mounted to a histological support according to an embodiment of the present disclosure. For clarity, the tissue sample is outlined using dashed lines.

FIG. 4: A detail view showing a cellular structure of the myocardial tissue identified in FIG. 2 by ‘C’.

FIG. 5: A process view diagram of use of a tissue support according to an embodiment of the present disclosure.

FIG. 6A: A perspective view of another embodiment of a tissue support according to an embodiment of the present disclosure.

FIG. 6B: A top view of the tissue support of FIG. 6A.

FIG. 7A: A perspective view of another embodiment of a tissue support according to an embodiment of the present disclosure.

FIG. 7B: A top view of the tissue support of FIG. 7A.

FIG. 8A: A perspective view of another embodiment of a tissue support according to an embodiment of the present disclosure.

FIG. 8B: A top view of the tissue support of FIG. 8A.

FIG. 9A: A perspective view of another embodiment of a tissue support according to an embodiment of the present disclosure.

FIG. 9B: A top view of the tissue support of FIG. 9A.

FIG. 10: A chart showing a method according to another embodiment of the present disclosure.

### DETAILED DESCRIPTION OF THE DISCLOSURE

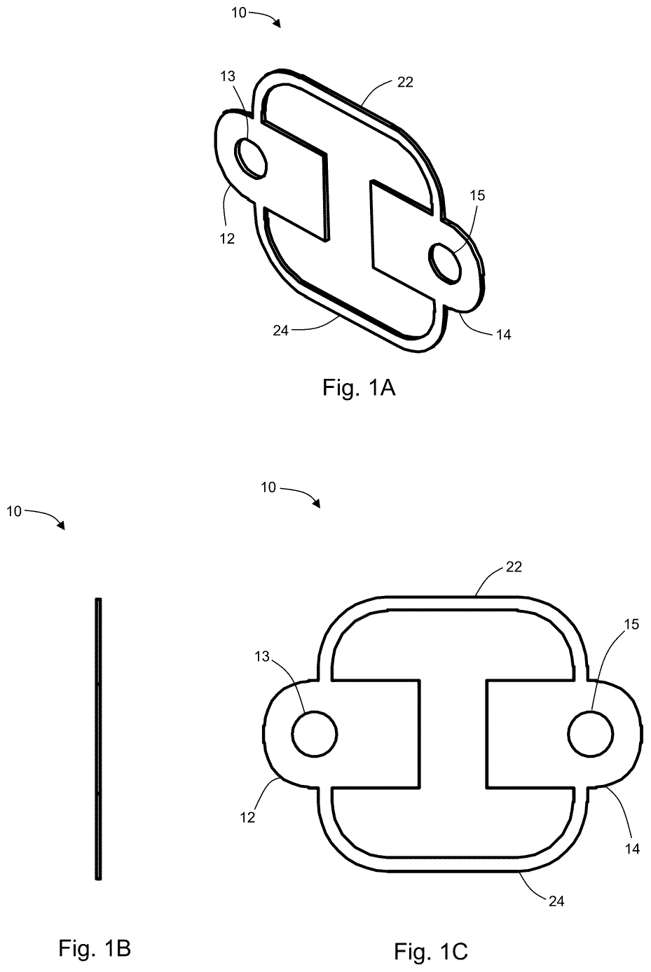

With reference to FIGS. 1A-1C, in a first aspect, the present disclosure may be tissue support 10 for securing a tissue sample, such as, for example, a myocardial tissue sample. In some embodiments, the tissue support may be a histological support. In some embodiments, the tissue sample may be dynamically secured, meaning that the tissue sample is secured using the tissue support 10 in a manner that allows convenient use in analysis equipment in which the tissue sample may be subjected to mechanical forces during testing. It should be noted that, although the present disclosure may describe myocardial tissue and testing, the tissue sample may be any type of tissue and the disclosure is not limited to myocardial tissue samples. For example, the tissue sample may be lung tissue, diaphragm tissue, etc.

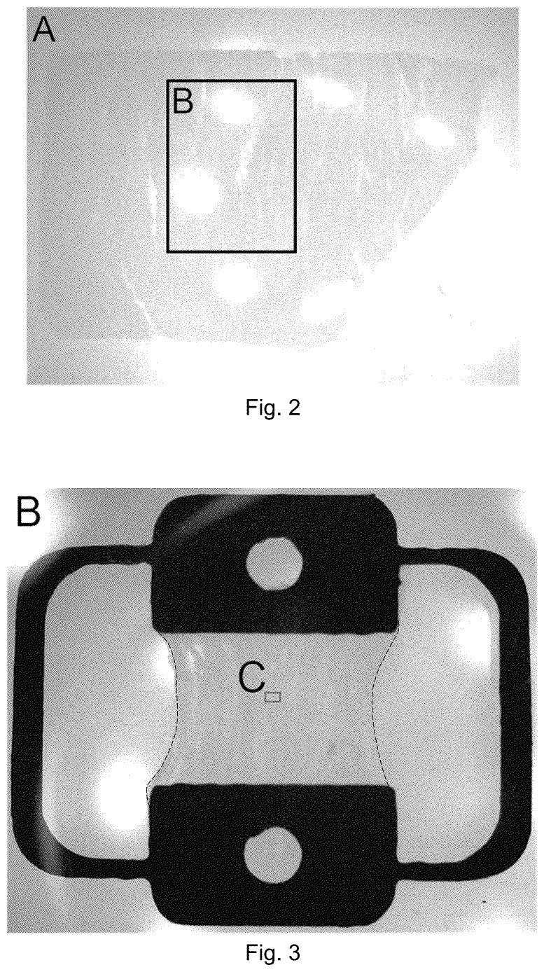

The tissue support 10 includes a first pad 12 for securing an end of the tissue sample and a second pad 14 for securing an opposite end of the tissue sample. The first pad 12 and the second pad 14 are spaced apart from each other by a predetermined distance. In this way, a portion of a mounted tissue sample extends between the first pad 12 and the second pad 14 (see, e.g., FIGS. 2 and 3). As used herein, the predetermined distance may be selected to hold the tissue sample at a desired length during mounting and handling, including, in some embodiments, a resting length of the tissue sample at the time of mounting.

The first pad 12 includes a first analyzer interface 13 configured to connect to a first corresponding interface of an analyzer, and the second pad 14 includes a second analyzer interface 15 configured to connect to a second corresponding interface of an analyzer. For example, the analyzer may be a cardiac slice machine. In such an analyzer, a tissue sample may be attached using clips which attach to posts of a force transducer and a length controller. In this example, the first analyzer interface 13 may be a hole through the first pad 12 configured to fit over a post of the force transducer/length controller, and the second analyzer interface 15 may be a hole through the second pad 14 configured to fit over another post of the force transducer/length controller. In this way, the analyzer may apply a force to the tissue sample (for example, a stretching force by increasing a distance between the first pad 12 and the second pad 14) and/or a length of the tissue sample may be measured. Other analyzer interface structures may include, without limitation, loops, notches, slots, hooks, or combinations of such structures.

The tissue support 10 includes a first severable spacing member 22 connecting the first pad 12 to the second pad 14. The first severable spacing member 22 maintains the spacing between the first pad 12 and the second pad 14 at the predetermined distance during mounting and handling. In this way, the tissue sample can be kept at a desired length (e.g., the length at the time of mounting to the tissue support 10). In some embodiments, an additional severable spacing member 24 connects the first pad 12 to the second pad 14 to maintain the predetermined distance.

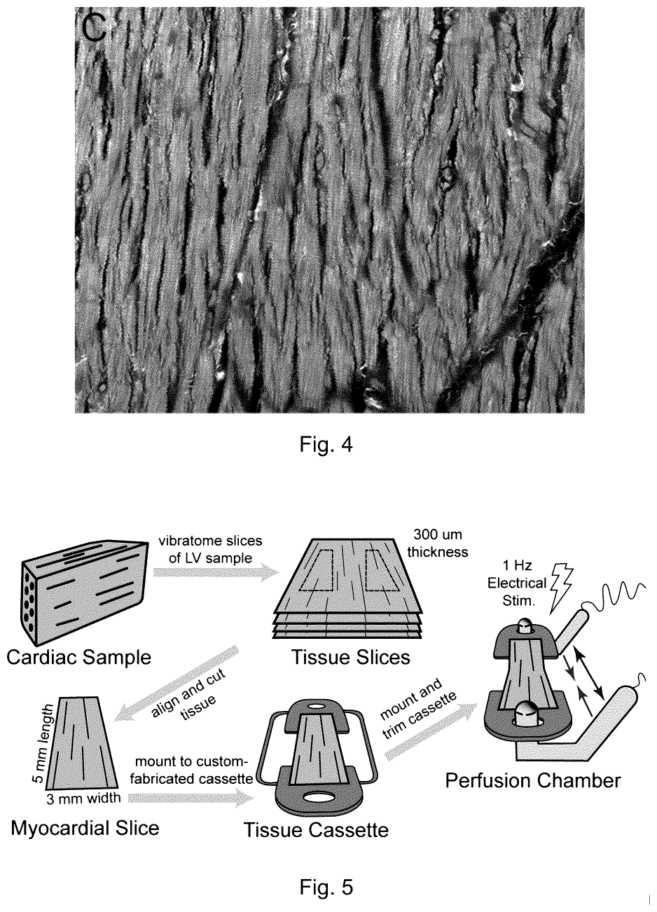

The first severable spacing member 22 is positioned at a first location so as to be readily severed while reducing the likelihood that a cutting tool intersects the tissue sample. For example, in the tissue support 10 of FIG. 4, the first severable spacing member 22 is spaced apart from the tissue sample (e.g., spaced apart from an axis extending between the first pad 12 and the second pad 14). FIG. 3 shows an example of the spacing between a tissue sample and severable spacing members showing how such a configuration allows a user to cut the severable spacing members with a low risk of a cutting tool reaching the tissue sample. Similarly, the additional severable spacing member 24, if present, may be positioned at a second location and spaced apart from a mounted tissue sample. In some embodiments, this spacing is described as a lateral offset from a region between the first pad 12 and the second pad 14 that receives the portion of the tissue sample extending between the pads, so that a cutting path to the severable member does not intersect that region.

In some embodiments, the first severable spacing member 22 and the additional spacing severable member 24, if present, are in a same plane as the first pad 12 and the second pad 14. In this way, the tissue support 10 may be fabricated by laser cutting from a sheet material, machining from a sheet material, 3D printing, or other techniques suited to forming the pads and severable members in a planar geometry.

The first severable spacing member 22 and the additional severable spacing member 24 may be configured to provide a force to counteract a response (e.g., change in length) of the tissue sample, thereby maintaining the predetermined distance between the first pad 12 and the second pad 14 prior to severing. For example, the first severable spacing member 22 and the additional severable spacing member 24 may be tuned by changing material, shape, size (e.g., cross-sectional area, overall length), other characteristics, or combinations of two or more of these characteristics. In a particular non-limiting example, the first severable spacing member 22 and the additional severable spacing member 24 may be configured to withstand a total force of 5 mN applied by a mounted tissue sample without a significant change in spacing between the first pad 12 and the second pad 14, where “significant” may be understood as not more than a 1%, 2%, 3%, 4%, 5%, or 10% change in the spacing depending on the test and analyzer. In some embodiments, one or more severable members may be configured as a flexure spring, serpentine spring, or accordion (zig-zag) spring.

In some embodiments, the tissue support 10 is a unitary structure formed from a single piece. In some embodiments, the tissue support 10 is assembled from two or more structures. The tissue support 10 may be made from a same material, such as a plastic or a composite. The tissue support 10 may be made from multiple materials; for example, the first pad 12 and the second pad 14 may be made from a first material and the first severable spacing member 22 and the additional severable spacing member 24 may be made from a second material different from the first material.

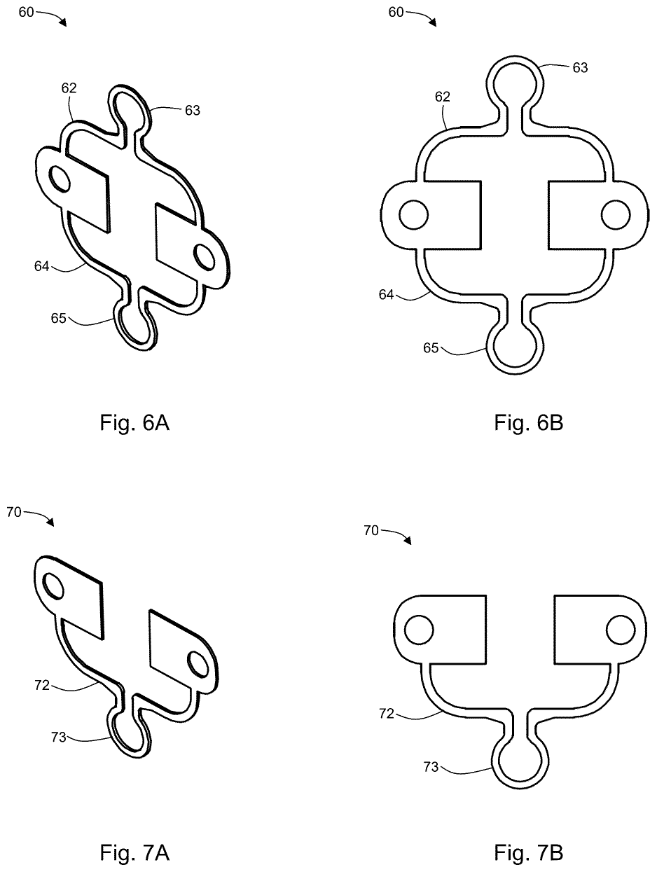

FIGS. 6A and 6B show another embodiment of a tissue support 60. In tissue support 60, each of the first and second severable members 62, 64 includes a loop 63, 65. The loops may serve various purposes. For example, such loops may be useful for holding and manipulating the tissue support by a user. The loop may alter the mechanical characteristics of the severable spacing members providing different resonance, etc. In another example, such a loop may provide for easier severability by a user. FIGS. 7A and 7B show an example of a tissue support 70 with only one severable spacing member 72 configured with a loop 73.

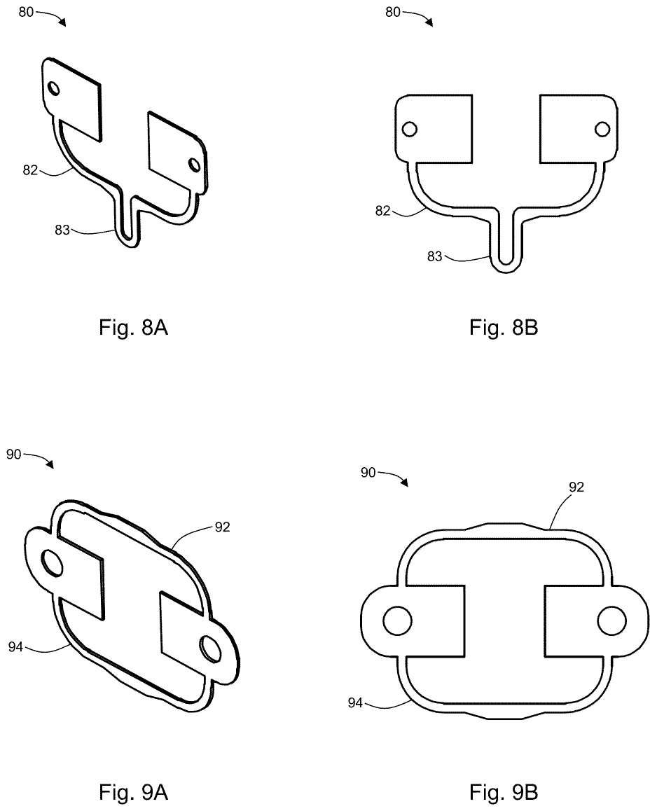

FIGS. 8A and 8B show another embodiment of a tissue support 80 that includes a first severable spacing member 82 with a U-shaped loop 83. FIGS. 9A and 9B show another embodiment of a tissue support 90 having a first several spacing member 92 and a second severable spacing member 94. The first and second several spacing members 92, 94 include portions which have a width greater than a width of the corresponding members of, for example, the tissue support 10 of FIGS. 1A-1C. These are illustrative examples, and other configurations of several spacing members may be used (other shapes, varying thicknesses, combinations of features, etc.) and are within the scope of the present disclosure.

The present disclosure relates to tissue supports that secure a tissue sample during handling and preparation, while also allowing the tissue sample to be coupled to analysis equipment and then released for mechanical loading once initial handling is complete. In some embodiments, a tissue support is used to hold a tissue sample at a selected length during mounting, transport, chemical exposure, and placement into an analyzer, and then the tissue support is modified by severing one or more connecting members so that the analyzer can apply mechanical forces to the tissue sample and measure tissue response.

In some embodiments, a tissue support has a first pad and a second pad that are spaced apart by a predetermined distance, with the tissue sample mounted so that a portion of the tissue sample extends between the pads. The predetermined distance may be selected to correspond to a desired length of the tissue sample during mounting, such as the resting length of the tissue sample at the time of attachment. The term “predetermined distance,” as used in this description, refers to a spacing established by the geometry of the tissue support before severing and maintained by one or more connecting members during mounting and handling. In some embodiments, the predetermined distance is selected before attachment by choosing a tissue support of a particular geometry, by selecting a support from a set of supports having different spacings, or by adjusting an adjustable support to a selected spacing and then fixing that spacing with one or more removable or severable members.

The pads may each include an analyzer interface configured to connect to corresponding fixation points of an analyzer. In some embodiments, each analyzer interface is formed as an aperture through the pad, sized and shaped to fit over a post of the analyzer so that the tissue support can be mounted to the analyzer by placing the apertures over the posts. In other embodiments, an analyzer interface may take other forms such as loops, notches, slots, hooks, or combinations thereof to engage a corresponding fixture or clamp of the analyzer. The analyzer may be a cardiac slice system or other tissue testing equipment that applies controlled forces to the tissue sample and/or measures length, displacement, or force during testing. For example, the analyzer may have a force transducer and a length controller that cooperate with the analyzer interfaces on the pads, allowing stretching or other mechanical loading by changing the spacing of the fixation points and allowing measurement of tissue length or tissue response.

In some embodiments, the first pad and second pad are connected by one or more severable spacing members that maintain the predetermined distance prior to severing. A severable spacing member is a structural element that holds the pads in a fixed spatial relationship during mounting and handling, and that can be severed by a user so that the pads become movable relative to one another for testing. In some embodiments, a first severable member extends between the pads, and in some embodiments a second severable member is also present, with the members located at different positions so that the pads remain held at the predetermined spacing prior to severing.

In some embodiments, the severable spacing member is positioned laterally offset from the region between the pads that receives the tissue sample. As used here, “laterally offset” refers to a configuration in which the severable member is spaced from an axis extending between the pads and from the tissue-receiving region so that a cutting operation directed to the severable member can be performed with reduced likelihood that the cutting tool intersects the tissue-receiving region. In one illustrated arrangement, the severable member is spaced away from the tissue sample outline shown between the pads so that a user can cut the severable member while maintaining the cutting tool away from the tissue sample. The tissue-receiving region may be understood as the volume or area between the pads in which the tissue sample portion spans from one pad to the other during mounting and prior to severing. The degree of offset may be selected based on the geometry of the tissue sample and the expected cutting tool; in some embodiments, the offset is selected so that a straight cutting path to the severable member does not intersect the tissue-receiving region. In some embodiments, the offset is selected so that a user can access the severable member from above the plane of the tissue support with scissors, a blade, or another cutting implement while keeping the implement outside the tissue-receiving region.

In some embodiments, one or more severable spacing members may be configured to provide a mechanical response that counters a response of the tissue sample, such as contraction or relaxation after extraction, thereby maintaining the predetermined distance between the pads during handling. This tuning may be achieved by selecting the material, shape, cross-sectional area, overall length, or other characteristics of the severable member. In a non-limiting example described in the disclosure, severable members are configured to withstand a total force on the order of 5 mN applied by a mounted tissue sample without a significant change in the pad spacing, where “significant” is contextual and may correspond to a change not more than about 1%, 2%, 3%, 4%, 5%, or 10% in various embodiments. In some embodiments, severable spacing members are shaped as flexure springs, including serpentine or accordion (zig-zag) geometries, allowing a desired stiffness while still being severable when testing is to begin. In some embodiments, severable members may include locally narrowed regions, notches, perforations, frangible webs, or other geometries that concentrate strain and facilitate severing using hand tools, while the remainder of the member provides the desired stiffness prior to severing. In some embodiments, severing is performed by cutting across a locally narrowed region, and in other embodiments severing is performed by tearing along a perforation line or breaking a frangible web by bending. In some embodiments, the severable member geometry is selected so that it remains stable under loads expected during handling and chemical exposure, yet can be severed without excessive force. In this disclosure, “gentle” handling or “gentle” severing force can be understood as forces that do not plastically deform the pads or damage the tissue sample attachment, and in some embodiments may correspond to user-applied forces achievable by handheld cutting tools without causing pad bending visible to the unaided eye; in some embodiments, the severable member is configured so that severing can be performed with a single cut of small scissors or a single slicing motion of a blade.

In some embodiments, the tissue support is planar, with the pads and severable members lying in a common plane. A planar configuration may facilitate fabrication from sheet material by laser cutting or machining, and may also facilitate additive manufacturing such as 3D printing, as described in the disclosure. In some embodiments, the tissue support is formed as a unitary structure from a single piece of material. In other embodiments, the tissue support is assembled from two or more structures. For example, pads may be formed as a first structure and one or more severable members may be formed as a second structure that is joined to the pads, allowing different material selection for different functions while maintaining the overall geometry of the tissue support. Joining may be performed using mechanical fasteners, adhesives, thermal bonding, overmolding, or other joining techniques suitable for the selected materials and for compatibility with biological workflows; in some embodiments, joining is selected to remain stable during chemical exposure associated with tissue preparation.

In some embodiments, the tissue support is made from a single material such as a plastic or composite. In other embodiments, the tissue support uses multiple materials, including embodiments in which the first and second pads are made from a first material and the severable spacing member or members are made from a second material different from the first material. The first material may be selected for stiffness, machinability, bonding to adhesives, or interaction with analyzer fixtures, and may include plastics, composites, or metals in various embodiments. The second material may be selected to provide a desired spring constant, to provide predictable severing behavior, or to accommodate fabrication of locally frangible regions. In some embodiments, the second material may be selected to be less stiff than the first material to allow a spring-like response, or more brittle than the first material to facilitate breaking at a frangible region, while still maintaining the predetermined distance prior to severing. In some embodiments, the pads are formed from a relatively rigid polymer and the severable members are formed from a polymer elastomer or a thin metallic spring material, with the selection tuned to the expected tissue forces.

The tissue support may be sized for the tissue samples of interest. In one example discussed in the disclosure, tissue samples may be on the order of 5 mm by 3 mm, and pad size and spacing may be selected accordingly. In additional embodiments provided to expand range support, tissue samples may be smaller or larger, and pad spacing may be selected across a range suited to biopsies from different organisms or different sample preparation workflows, including small animal and larger animal samples, as described. In some embodiments, pad-to-pad spacing is selected within a range from about 1 mm to about 50 mm, inclusive (e.g., about 2 mm, 5 mm, 10 mm, 20 mm, or 30 mm), depending on the analyzer geometry and the tissue sample size. In some embodiments, pad thickness is selected within a range from about 0.1 mm to about 5 mm, inclusive (e.g., about 0.25 mm, 0.5 mm, 1 mm, 2 mm, or 3 mm). In some embodiments, pad lateral dimensions are selected within a range from about 1 mm to about 30 mm, inclusive (e.g., about 2 mm, 5 mm, 10 mm, 15 mm, or 20 mm). In some embodiments, apertures used as analyzer interfaces are selected to fit posts of the analyzer, and the aperture diameter may be selected within a range from about 0.25 mm to about 10 mm, inclusive (e.g., about 0.5 mm, 1 mm, 2 mm, 3 mm, 5 mm, or 8 mm).

The pads may secure the tissue sample using adhesive, mechanical engagement, or both. In some embodiments, the tissue sample is attached to each pad using a biocompatible cyanoacrylate adhesive, including histo-acrylate or Vetbond, as examples. In some embodiments, the tissue support material is selected to bond with the adhesive; the disclosure describes an example in which the tissue support includes a polyacrylate that crosslinks with cyanoacrylate adhesive. In additional embodiments provided here to expand enablement, a user may apply a controlled amount of adhesive to each pad, position an end of the tissue sample on the adhesive, and allow the adhesive to set while the tissue sample is held at the predetermined distance by the severable spacing member. In some embodiments, the adhesive is applied as a thin layer to reduce wicking into the tissue and to maintain a defined attachment area. In some embodiments, the adhesive layer thickness is selected within a range from about 1 μm to about 500 μm, inclusive (including, for example, about 5 μm, 10 μm, 50 μm, 100 μm, and 250 μm), depending on tissue type and desired bond area.

In some embodiments, at least one pad has a gripping surface configured to secure the tissue sample, such as micro surface features including protrusions, hooks, or barbs. Such microfeatures may be formed by molding, machining, laser texturing, or additive manufacturing, and may be arranged to increase frictional engagement, provide mechanical interlock with the tissue surface, or increase effective bonding area for adhesives. In additional embodiments provided here to expand written description, microfeatures may have heights within a range from about 5 μm to about 2 mm, inclusive (including, for example, about 10 μm, 50 μm, 100 μm, 250 μm, 500 μm, and 1 mm), and may be distributed with spacings within a range from about 5 μm to about 5 mm, inclusive (including, for example, about 25 μm, 100 μm, 250 μm, 500 μm, 1 mm, and 2 mm). In some embodiments, microfeatures are oriented to resist pullout along the axis between pads when the tissue sample is loaded in the analyzer.

In some embodiments, the tissue support is electrically conductive, and an adhesive used for mounting is also electrically conductive, allowing direct electrical stimulation of the tissue sample and/or recording of tissue action potentials through the tissue support. The disclosure describes an example of conductive adhesive using carbon nanotube-doped histoacrylate adhesive. In some embodiments, electrical stimulation is performed after severing the spacing member or members, when the pads are independently movable under analyzer control, and electrical current can be delivered through the tissue sample via the conductive pads. In additional embodiments provided here to expand enablement, conductive portions may be limited to regions near the tissue attachment areas to reduce unintended current paths through the severable members, or conductive paths may be routed through the support so that stimulation electrodes are positioned near the tissue ends. In some embodiments, the tissue support interfaces with an electrical stimulation or recording module of the analyzer, for example by conductive contact surfaces on the pads that touch conductive fixtures of the analyzer.

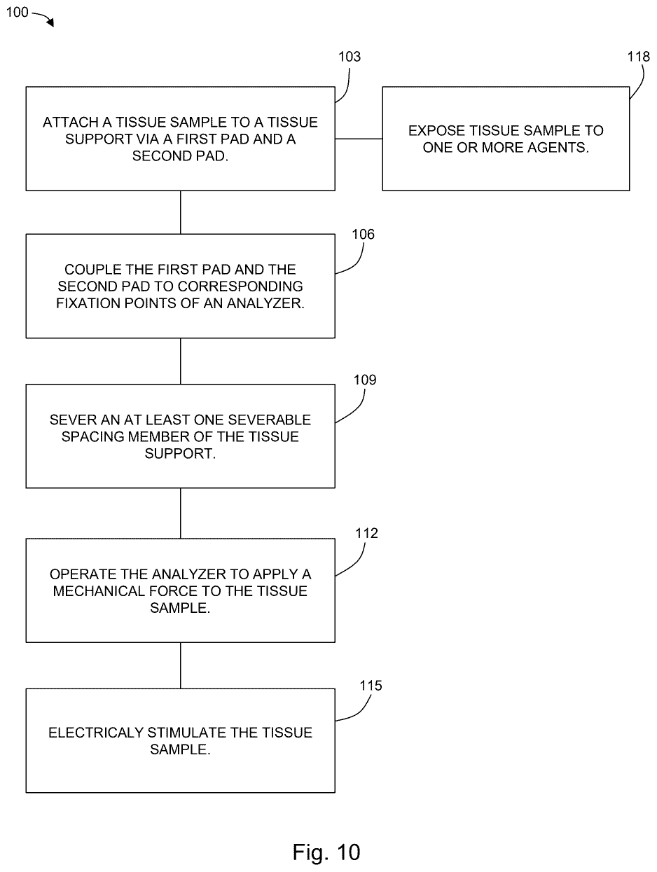

With respect to FIG. 10, a method 100 of preparing a tissue sample for analysis may use any of the tissue supports described above. In some embodiments, a first end of the tissue sample is attached 103 to the first pad and a second end of the tissue sample is attached to the second pad while the severable spacing member maintains the predetermined distance between pads. The tissue sample may be attached using a biocompatible adhesive such as cyanoacrylate, and the tissue support material may be selected to bond with the adhesive, including embodiments using polyacrylate to promote crosslinking with cyanoacrylate. In some embodiments, the tissue sample may be exposed 118 to one or more chemical agents. For example, the tissue sample may be prepared for storage and/or testing while mounted to the tissue support by exposure to chemical agents. The tissue support, with the attached tissue sample, is then coupled 106 to the analyzer by engaging the analyzer interfaces of the pads with fixation points of the analyzer. In some embodiments, coupling is performed by placing pad apertures over analyzer posts so that the tissue support is seated on the analyzer. After coupling, each severable spacing member is severed 109 so that the pads are movable relative to one another, allowing the analyzer to apply mechanical force to the tissue sample and to measure tissue response, such as force and length or displacement. In embodiments where the severable member is laterally offset from the tissue-receiving region, severing may be performed by cutting the laterally offset member with reduced likelihood of contacting the tissue sample. The analyzer is operated 112 to apply a mechanical force to the tissue sample via the first pad and the second pad. In some embodiments, the analyzer applies a stretching force by increasing the distance between fixation points, and the analyzer measures the applied force and/or tissue length using a force transducer and length controller. In embodiments with conductive portions, the method may further include electrically stimulating 115 the tissue sample or recording electrical signals through the tissue support, including embodiments in which current flows through the tissue sample between conductive pads.

Although particular embodiments are described with reference to myocardial tissue and cardiac slice testing, the disclosure indicates that the tissue sample may be other tissue types such as lung tissue or diaphragm tissue, and the tissue support may be used with different analyzers and workflows that benefit from maintaining tissue orientation and length during handling and then enabling mechanical loading during testing. In some embodiments, the tissue support may be used in workflows that involve repeated chemical exposures, rinses, temperature changes, or imaging steps while maintaining a defined tissue length, after which severing allows mechanical cycling or measurement. In some embodiments, the predetermined distance is selected to reduce artifacts introduced by post-extraction contraction by holding the tissue sample near a selected length during early handling, and severing is performed only after the tissue sample is secured to the analyzer and ready for controlled loading.

Although the present disclosure has been described with respect to one or more particular embodiments, it will be understood that other embodiments of the present disclosure may be made without departing from the spirit and scope of the present disclosure.

What is claimed is:

1. A tissue support for securing a tissue sample for analysis, comprising:
a first pad having a first analyzer interface;
a second pad having a second analyzer interface, the second pad being spaced from the first pad by a predetermined distance such that, when the tissue sample is mounted to the first pad and the second pad, a portion of the tissue sample extends between the first pad and the second pad; and
at least one severable spacing member coupling the first pad to the second pad and configured to maintain the predetermined distance while the tissue sample is mounted, wherein the at least one severable spacing member is positioned laterally offset from a region between the first pad and the second pad that is configured to receive the portion of the tissue sample.

2. The tissue support of claim 1, wherein the at least one severable spacing member comprises a first severable spacing member and a second severable spacing member, each coupling the first pad to the second pad at respective different locations.

3. The tissue support of claim 1, wherein the first analyzer interface and the second analyzer interface each comprise an aperture through the respective pad.

4. The tissue support of claim 1, wherein the at least one severable spacing member includes a reduced-thickness section, notch, perforation, or frangible web configured to facilitate severing.

5. The tissue support of claim 1, wherein the at least one severable spacing member is configured as a flexure spring having a serpentine spring geometry or an accordion spring geometry.

6. The tissue support of claim 1, wherein the first pad, the second pad, and the at least one severable spacing member are coplanar, and wherein the tissue support is formed from sheet material.

7. The tissue support of claim 1, wherein at least one of the first pad or the second pad comprises a gripping surface having microfeatures configured to engage the tissue sample.

8. The tissue support of claim 1, wherein at least one of the first pad or the second pad is configured to bond to a biocompatible cyanoacrylate adhesive used to attach the tissue sample to the pad.

9. The tissue support of claim 8, wherein at least one of the first pad or the second pad comprises a polyacrylate configured to crosslink with the cyanoacrylate adhesive.

10. The tissue support of claim 1, wherein the tissue support comprises an electrically conductive portion configured such that electrical current can be delivered to, or sensed from, the tissue sample via the tissue support after the at least one severable spacing member is severed.

11. The tissue support of claim 1, wherein the first pad and the second pad comprise a first material, and the at least one severable spacing member comprises a second material different from the first material.

12. A tissue analysis system, comprising:
the tissue support of claim 1; and
an analyzer having a first fixation point configured to engage the first analyzer interface and a second fixation point configured to engage the second analyzer interface,
wherein, after severing the at least one severable spacing member, the analyzer is configured to apply a mechanical force to the tissue sample via the first pad and the second pad and to measure a response of the tissue sample.

13. The tissue analysis system of claim 12, wherein the analyzer comprises a force transducer and a length controller configured to measure a length change of the tissue sample while applying the mechanical force.

14. The tissue analysis system of claim 12, wherein the first fixation point and the second fixation point comprise posts and the first analyzer interface and the second analyzer interface comprise apertures sized to fit over the posts.

15. The tissue analysis system of claim 12, wherein the analyzer further comprises an electrical stimulation or recording module electrically coupled to the tissue support.

16. A method of preparing a tissue sample for analysis, comprising:
attaching a first end of the tissue sample to a first pad of a tissue support and attaching a second end of the tissue sample to a second pad of the tissue support such that a portion of the tissue sample extends between the first pad and the second pad while a predetermined distance between the first pad and the second pad is maintained by at least one severable spacing member coupling the first pad to the second pad;
coupling the first pad to a first fixation point of an analyzer and coupling the second pad to a second fixation point of the analyzer;
severing the at least one severable spacing member; and
after severing, operating the analyzer to apply a mechanical force to the tissue sample via the first pad and the second pad.

17. The method of claim 16, wherein attaching comprises using a biocompatible cyanoacrylate adhesive, and wherein at least one of the first pad or the second pad comprises a polyacrylate configured to bond with the cyanoacrylate adhesive.

18. The method of claim 16, further comprising, prior to severing, exposing the tissue sample to one or more chemical agents to prepare the tissue sample for storage or testing.

19. The method of claim 16, wherein severing comprises cutting a laterally offset portion of the tissue support that is spaced from the portion of the tissue sample extending between the first pad and the second pad.

20. The method of claim 16, further comprising electrically stimulating the tissue sample or recording an electrical signal from the tissue sample through an electrically conductive portion of the tissue support.
